(19) United States
(12) Patent Application Publication
(10) Pub. No.: US 2002/0035058 A1
(43) Pub. Date: Mar. 21, 2002

(54) Isopentenyl pyrophosphate isomerase (IPI) and/or prenyl transferase inhibitors

(75) Inventors: Richard John Brown, Sheffield (GB); Donald Jeremy Watts, Sheffield (GB); Robert Graham Goodwin Russell, Sheffield (GB); Michael John Rogers, Slains (GB)

Correspondence Address: Candice J., Clement, Esq., Heslin Rothenberg Farley & Mesiti P.C., 5 Columbia Circle, Albany, NY, 12203, US

(73) Assignee: The University of Sheffield, Sheffield (GB)

(21) Appl. No.: 09/950,095

(22) Filed: Sep. 10, 2001

Related U.S. Application Data
(63) Continuation of application No. 09/660,642, filed on Sep. 13, 2000, now abandoned.
Application No. 09/660,642 is a continuation of application No. 09/180,814, filed on Jan. 27, 1999, now abandoned.
Application No. 09/180,814 is a 371 of international application No. PCT/GB97/01302, filed on May 12, 1997.

(30) Foreign Application Priority Data
Apr. 25, 1997 (GB) 9708329.9
May 15, 1996 (GB) 9610174.6

(51) Int. Cl. A61K 31/00

(52) U.S. Cl. 514/1

(57) ABSTRACT

Methods of treatment and prophylaxis of various diseases and disorders, and in particular diseases and disorders of lipid and bone metabolism, involving the administration of prenyl transferase (farnesyl pyrophosphate synthase) and/or isopentenyl pyrophosphate isomerase inhibitor compounds are disclosed.

### FIELD OF THE INVENTION

The present invention relates to prenyl transferase (farnesyl pyrophosphate synthase) and isopentenyl pyrophosphate isomerase (IPI) inhibitors, and to the use of such inhibitors in various forms of therapy and prophylaxis. In another aspect, the present invention relates to the field of bone metabolism (e.g. bone resorption), and in particular to the use of prenyl transferase and IPI inhibitors in the treatment of various diseases and disorders of bone metabolism and to the use of prenyl transferase in the screening, isolation, synthesis and evaluation of osteoactive drugs.

### BACKGROUND TO THE INVENTION

Lipid Metabolism

Lipids occur in the blood mainly as cholesterol and triglycerides, with smaller amounts of phospholipids, fatty acids, and fatty acid esters. In vivo, cholesterol and triglycerides are complexed with proteins (known as apolipoproteins) and transported in the form of lipoprotein particles. The lipoprotein particle surface is composed largely of phospholipid, free cholesterol and protein, and the core contains mostly triglyceride and cholesterol esters.

Lipoprotein particles are divided into four major groups on the basis of density, composition and electrophoretic mobility. Chylomicrons are formed in the intestine and are large triglyceride-rich particles derived from dietary fat. Very low density lipoproteins (hereinafter VLDLs) are composed largely of endogenous triglycerides and are synthesised in the liver. Low density lipoproteins (hereinafter LDLs) are rich in cholesterol and are mainly end products of VLDL catabolism. High density lipoproteins (HDLs) contain about 50% protein, are produced in the liver and intestine and act as acceptors of lipids, especially free cholesterol.

Hyperlipidaemia (or hyperlipoproteinaemia) embraces a group of disorders associated with elevated levels of lipids in the blood. Hyperlipidaemias may be inherited or secondary to dietary factors, excessive alcohol intake or primary disease states (such as hypothyroidism and diabetes mellitus), and can also arise as a side effect of various drug treatments (e.g. the administration of beta-blockers, corticosteroids, oestrogens, oral contraceptives or thiazide diuretics). Other factors affecting the lipid composition of the blood include physical activity, age and sex.

Six types of hyperlipoproteinaemia have been recognized on the basis of the patterns of the particular lipoproteins that are elevated. They are:

Type I (hyperchylomicronaemia) characterised by the presence of chylomicrons and by normal or only slightly increased concentrations of very low-density lipoproteins;

Type IIa (hyper-β-lipoproteinaemia) characterised by an elevation in the concentration of low-density lipoproteins;

Type IIb characterised by an elevation in the concentration of low-density lipoproteins and of very low-density lipoproteins;

Type III (“floating β” or “broad β” pattern) characterised by the presence of very low-density lipoproteins having an abnormally high cholesterol content And an abnormal electrophoretic mobility;

Type IV (hyperpre-β-lipoproteinaemia) characterised by an elevation in the concentration of very low-density lipoproteins, but no increase in the concentration of low-density lipoproteins, and by the absence of chylomicrons;

Type V (hyperpre-β-lipoproteinaemia and chylomicronaemia) characterised by an elevation in the concentration of very low-density lipoproteins and the presence of chylomicrons.

Primary hyperlipidaemias may also be classified according to the genetic and metabolic disorder resulting in the following categories:

(a) familial hypercholesterolaemia, which is usually heterozygous but very rarely may be homozygous, characterised by a type IIa pattern (occasionally a type IIb pattern is present);

(b) familial hypertriglyceridaemia, usually associated with a type IV or type V pattern;

(c) familial combined hyperlipidaemia, characterised by elevated cholesterol only, elevated triglyceride only, or elevated cholesterol and triglyceride—type IIa, type IV, or type IIb patterns may be found;

(d) familial dysbetalipoproteinaemia (remnant hyperlipoproteinaemia or broad-βdisease) showing the type III pattern; and

(e) lipoprotein lipase deficiency or apolipoprotein C-II deficiency showing a type I or type V pattern.

Many hyperlipidaemias are associated with an increased risk of atherosclerosis, ischaemic hears disease and related complications. Thus, hypolipidaemic agents can find application in the treatment and prophylaxis of a wide range of cardiovascular disorders associated with hyperlipidaemia.

Known treatments for the hyperlipidaemias described above include bile acid sequestrants (colestipol hydrochloride and cholestyramine), niacin, probucol, gemfibrozil and neomycin sulphate. However, unpleasant side effects often prevent the use of these agents.

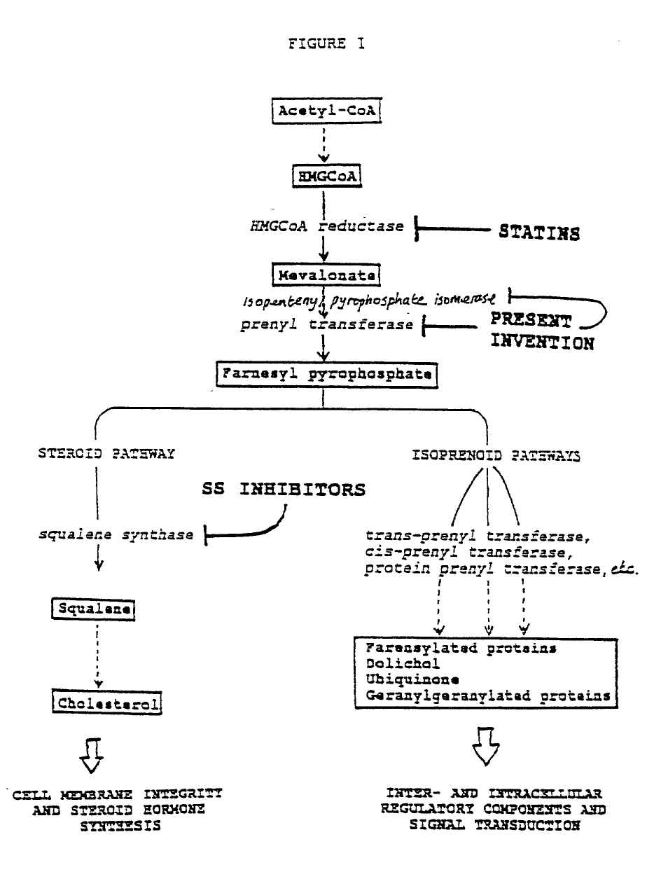

Another class of hypolipidaemics are the “statins” (Hoeg and Brewer, 1987, JAMA, Vol. 258 (24), pages 3532-3536). These compounds act at the beginning of the cholesterol biosynthetic pathway (see FIG. 1) by competitively inhibiting 3-hydroxy-3-methylglutaryl coenzyme A reductase (herinafter HMGCoAR, the rate determining enzyme for cholesterol synthesis). All of the statins presently in use are HMG-CoA analogues, and include mevastatin (compactin), lovastatin (mevinolin), prevastatin (eptastatin) and simvastatin (synvinolin)

The statins work by increasing the catabolism of LDL as well as decreasing its synthesis. LDL particles contain apolipoprotein B (apoB), and are removed From circulation by means of high affinity LDL receptors which recognize apoB. These receptors are upregulated by the cholesterol depletion induced by the statins. Thus, the compensatory responses following the block of cholesterol synthesis are central to the efficacy of the statins: patients lacking LDL receptors fail to respond to statin administration (see below).

However, the use of HMGCoAR inhibitors as hypolipidaemic agents is associated with several problems. Adverse reactions include gastrointestinal disturbances, headache, rash and pruritus. Liver damage can occur in hepatocompromized subjects, as well as myositis with increased values for creatine phosphokinase. Moreover, severe muscle effects (including myolysis, rhabdomyolysis, myositis and myolitis) have seen reported, particulary in hepatocompromized patients or in patients in which the metabolism of the inhibitor is impaired (such as patients receiving gemfibrozil or cyclosporin). The inhibitors may also interfere with DNA replication and there are reports of carcinogenicity and teratogenicity.

A further problem associated with the statins is the failure of certain classes of patients to respond. Nonresponders can amount to 20% of the target patient population, not including those patients lacking functional LDL receptors (such as those homozygous for familial hypercholesterolaemia) which also fail to respond.

Some workers have ascribed certain side effects to the fact that the metabolic block induced by the statins effectively shuts down the isoprenoid biosynthetic pathways which lead from farnesyl pyrophosphate to a large number of different products (including ubiquinone and dolichol). Since many of these products are essential for cell viability, it has been suggested that at least some of the side effects associated with the statins might arise from the block imposed on the isoprenoid pathways rather than from that on cholesterol biosynthesis per se.

Attempts have therefore been made to block cholesterol synthesis more specifically by using inhibitors of squalene synthase. This enzyme catalyzes the reductive dimerization of farnesyl pyrophosphate to form squalene at the final branch point of the cholesterol biosynthetic pathway (see FIG. 1). Since the enzyme is unique to this pathway, selective inhibitors of squalene synthase might be expected to reduce cholesterol levels without eliminating the pool of essential branch products of the isoprene pathway (such as dolichol, coenzyme-Q and prenylated proteins). Accordingly, it has been suggested that squalene synthase inhibitors could have advantages over the statins as hypolipidaemics. On the basis of this rationale, several different types of squalene synthase inhibitors have been isolated or synthesized (Ciosek et al. (1993) J. Biol. Chem. Vol. 268 (33), pages 24832-24837 and Amin et al. (1992) J. Lipid Res. Vol. 33, pages 1657-1663). Some of the known squalene synthase inhibitors are lipophilic bisphosphonates.

However, it has become evident that important beneficial effects of the statins (particularly those associated with the alleviation of cardiovascular disease events associated with hyperlipidaemia) may arise from their action on mevalonate pathway products other than cholesterol, particularly the isoprenoids (Massy et al., 1996, Lancet, Vol. 347 pages 102-103). It has also been suggested that effective inhibition of the cholesterol biosynthetic pathway depends to a significant extent on negative feedback arising from suppression of other isoprenoid pathways.

Thus, the squalene synthase inhibitors may not be as effective as the statins in alleviating cardiovascular disease attendant on hyperlipidaemia, and may be less effective in lowering cholesterol levels.

There is therefore a need for hypolipidaemics which exhibit reduced side effects and increased efficacy and breadth of application.

Bone Diseases and Disorders

A number of diseases are recognized which arise from bone destruction or disorders of bone metabolism. These diseases are of great clinical importance and have been the subject of intense scientific research for many decades.

Bone destruction can result from various cancers and also from rheumatoid arthritis. Metabolic bone disorders commonly involve excessive bone resorption and include Paget's disease, hypercalcaemia (both tumour-induced and non-tumour induced), bone metastases and osteoporosis.

Paget's disease (a focal increase in bone turnover) is fairly common, and in some countries affects up to 5% of the population over 50 years of age. The disease may be caused by a slow virus infection, and leads to bone pain, deformities and fractures.

Bone metastases can induce bone destruction either through local invasion or via the secretion of bone-resorbing agents into the blood stream.

Hypercalcaemia can result either from an increase in the flow of calcium from bone or the intestine to the blood, or from an increase in tubular reabsorption of calcium in the kidney. It can induce a wide variety of physiological disturbances and can be life threatening.

Osteoporosis is characterized by a reduction in the quantity of bone leading to atrophy of skeletal tissue. This loss of bone tissue often causes mechanical failure, and bone fractures frequently occur in the nip and spine of women suffering from postmenopausal osteoporosis. Kyphosis (abnormally increased curvature of the thoracic spine) is another common symptom.

Two types of osteoporosis are recognized: primary and secondary. Secondary osteoporosis is the result of an identifiable disease process or agent, while primary osteoporosis (which constitutes about 90% of all cases) includes postmenopausal osteoporosis, age-associated osteoporosis (affecting a majority of individuals over the age of 70) and idiopathic osteoporosis affecting middle-aced and younger men and women.

The mechanism of bone loss in osteoporosis is believed to involve an imbalance in the process of “bone remodelling”. Bone remodeling occurs throughout life, renewing the skeleton and maintaining the strength of bone. This remodelling involves the erosion and filling of discrete sites on the surface of bones by an organized group of cells called “basic multicellular units” or “BMUs”. BMUs primarily consist of osteoclasts, osteoblasts, and their cellular precursors. In the remodelling cycle, bone is resorbed at the site of an “activated” BMU by an osteoclast, forming a resorption cavity. This cavity is then filled with bone by osteoblasts.

Normally, in adults, the remodelling cycle results in a small deficit in bone, due to incomplete filling of the bone resorption cavity. Thus, even in healthy adults, age-related bone loss occurs. However, in many people, particularly in postmenopausal osteoporotics, there is an increase in the number of BMUs that are activated. This increased activation accelerates bone remodelling, resulting in abnormally high bone loss.

Many compositions and methods are described in the medical literature for the treatment of the above-described diseases and conditions, and most attempt to either slow the loss of bone or produce a net gain in bone mass.

Administration of oestrogen has been used as a means both to prevent and to treat osteoporosis in postmenopausal women. However, the use of oestrogen has been associated with certain side effects, such as uterine bleeding. Other treatments are based on the administration of parathyroid hormone.

The hormone calcitonin has also been used to treat Paget's disease (and to a lesser extent tumour bone disease), and can be effective in decreasing bone turnover. However, the incidence of relapse is high, and side effects on the vascular system limit the therapeutic usefulness of calcitonin.

One of the most successful class of drugs for the treatment of the above diseases has proved to be the bisphosphonates.

It has long been known that inorganic pyrophosphate and polyphosphate (PPi) have high affinity for bone mineral and are able to inhibit the precipitation and dissolution of calcium phosphate crystals in vitro and to inhibit bone mineralization in vivo. These activities are thought to arise from direct physicochemical effects (such as adsorption to hydroxyapatite, inhibition of dissolution of hydroxyapatite and crystal growth inhibition) Pyrophosphates have therefore found application as bone imaging agents for diagnostic purposes (usually in the form of “technetium derivatives) and as antitartar agents for use in toothpastes.

However, inorganic pyrophosphates are rapidly hydrolysed following administration (particularly oral administration) due to the presence of the relatively labile phosphorus-oxygen bond P—O—P. This severely limits their pharmaceutical utility, and has prompted a search for PPi analogues which exhibit similar physicochemical activities while resisting enzymatic hydrolysis 7 in vivo.

Bisphosphonates, which are characterized by phosphorus-carbon (P—C—P) bonds, are stable analogues of naturally occurring inorganic pyrophosphates which to a great extent overcome the limitations associated with inorganic pyrophosphates. Bisphosphonates are resistant to chemical and enzymatic hydrolysis but retain the therapeutic activity of PPi.

Unlike pyrophosphates, however, bisphosphonates exhibit properties which extend beyond those attributable to purely physicochemical phenomena. In particular, bisphosphonates have been found to be inhibitors of osteoclast-mediated bone resorption in organ cultures of bone and in animal models. Bisphosphonates therefore have broader clinical utility than PPi, and have found widespread application in four main clinical areas: (a) as bone imaging agents for diagnostic purposes, usually in the form of “technetium derivatives, (b) as anti-resorptive agents to combat bone loss associated with Paget's disease, hypercalcaemia associated with malignancy and bone metastases or osteoporosis, (c) as calcification inhibitors in patients with ectopic calcification and ossification, and (d) as antitartar agents for use in toothpastes.

Bisphosphonates are now among the most important therapeutic agents for the treatment of pathological disorders of bone metabolism, including osteoporosis. Moreover, since some bisphosphonates appear to have anti-inflammatory as well as anti-resorptive effects in vivo, they may also have utility in the treatment of inflammation and rheumatoid arthritis.

However, despite widespread recognition of the importance of bisphosphonates as a class of anti-resorptive drugs, the mechanisms of action of these compounds remain obscure. It has been suggested that bisphosphonates may affect the differentiation and recruitment of osteoclast precursors or alter the capacity of mature osteoclasts to resorb bone by altering the permeability of the osteoclast membrane to small ions. Another hypothesis is that they act by affecting lysosomal enzyme production or cell metabolism or through toxic effects on osteoclasts. A further suggestion is that bisphosphonates affect other cells in the bone microenvironment that regulate the activity of the osteoclasts that are involved in the antiresorptive mechanism.

As is clear from the above discussion, the bisphosphonates represent a important class of drug which has opened up new approaches in the therapy of bone diseases. However, as a class the bisphosphonates are characterized by poor intestinal absorption and the ideal bisphosphonate would show substantial and consistent intestinal absorption, consistent and reversible effects on bone turnover, low toxicity and (for appropriate treatment regimens) shortened residence time in bone. Present bisphosphonates fulfil these ideals only to a limited degree. Moreover, there exists considerable scope for further innovation and development and many further clinical applications may exist requiring different profiles of activity.

There is therefore a need to understand the mechanism of action of the bisphosphonates, to rapidly and efficiently screen a large number of potential improved bisphosphonate drugs and to identify and create compounds having improved therapeutic activity.

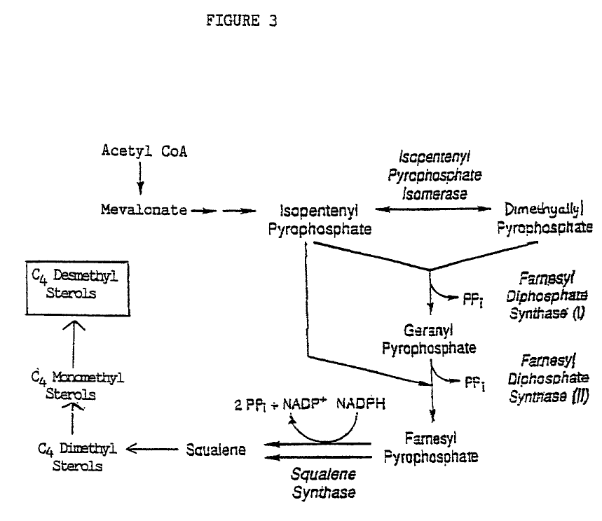

The present inventors have now discovered that bisphosphonate drugs exert their effect (at least in part) via inhibition of prenyl transferase and/or IPI. As explained above, prenyl transferase catalyzes the condensation of isopentenyl pyrophosphate with an allylic pyrophosphate at a point in the sterol biosynthetic pathway just prior to the branchpoint into the sterol and isoprenoid pathways (see FIGS. 1 and 3).

Isopentenyl pyrophosphate isomerase (IPI) catalyses interconversion of isopentenyl pyrophosphate and dimethylallyl pyrophosphate (FIG. 3) via a carbocation intermediate.

### SUMMARY OF THE INVENTION

According to the present invention there are provided IPI and/or prenyl transferase inhibitors (ie inhibitors of IPI and/or prenyl transferase) for use in various forms of therapy and prophylaxis.

For example, the IPI and/or prenyl transferase inhibitors of the present invention find particular utility in the modulation of lipid metabolism, cell proliferation, isoprenoid-related cellular apoptosis and cellular signal transduction. The IPI and/or prenyl transferase inhibitors are also useful as fungicides or herbicides. The inhibitors may have dual inhibitory activity, and inhibit both IPI and prenyl transferase. They may also inhibit other isoprenoid synthetic enzymes.

The present invention also provides a new class of antiresorptive (osteoactive) drugs based on the presence of IPI and/or prenyl transferase inhibitory activity, and provides methods for rapidly and efficiently screening a large number of potential osteoactive drugs (e.g. improved osteoactive bisphosphonates) and for the identification and synthesis of compounds having improved antiresorptive and/or anti-inflammatory activity.

Prenyl Transferase Inhibitors, IPI Inhibitors and Inhibitors of IPI and/or Prenyl Transferase, Lipid Metabolism, Cell Proliferation, Apoptosis and Signal Transduction

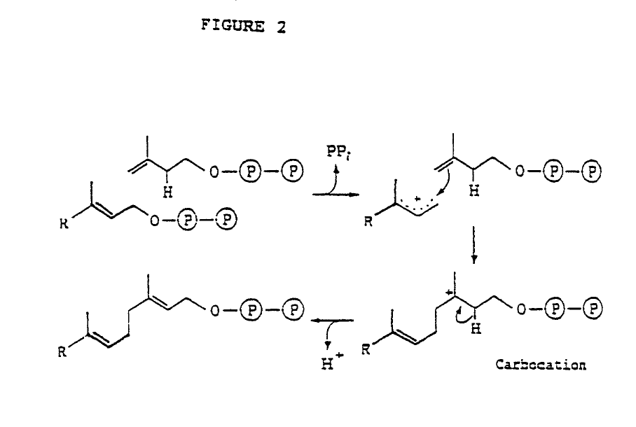

Prenyl transferase catalyzes the condensation of isopentenyl pyrophosphate with an allylic pyrophosphate, and the prenyl transferase inhibitors of the present invention are inhibitors of this enzyme. It is specific for isopentenyl pyrophosphate but can use either the 5-carbon dimethylallyl pyrophosphate (farnesyl diphosphate synthase I) or the 10 carbon geranyl pyrophosphate (farnesyl disphosphate synthase II) as its allylic substrate. The prenyl transferase-catalyzed condensation mechanism proceeds via a carbocation intermediate, as shown in FIG. 2.

The site of action of the IPI and prenyl transferase inhibitors of the present invention is shown in FIG. 1, along with those of the known HMGCoA reductase and squalene synthase inhibitors. It can be seen that the IPI and/or prenyl transferase inhibitors of the present invention act at a point in the cholesterol biosynthetic pathway before the synthesis of farnesyl pyrophosphate. In contrast, the known squalene synthase inhibitors act after the synthesis of farnesyl pyrophosphate. Thus, like the statins, the IPI and/or prenyl transferase inhibitors of the invention are upstream inhibitors, in that they inhibit both the cholesterol biosynthetic pathway and the various isoprenoid pathways.

However, the IPI and/or prenyl transferase inhibitors of the invention act at the step immediately preceding the branch point into the various cholesterol/isoprenoid pathways. This has important consequences with respect to the nature of the perturbations induced in the pools of mevalonate metabolites, as explained below.

HMGCoA reductase mediates the rate-determining step of cholesterol biosynthesis and is the most elaborately regulated enzyme of the cholesterol biosynthetic pathway. It is subject to control by both competitive and allosteric mechanisms, by phosphorylation and dephosphorylation as well as by long-term regulation. Cholesterol and other metabolites are feedback regulators of the enzyme. Thus, inhibition of HMGCoA reductase profoundly perturbs the sizes of a large number of different metabolite pools, and some of the side effects associated with the use of the statins arise from these perturbations. Moreover, the reduction of cholesterol levels relieves negative feedback on the HMGCoA reductase and results in a large increase in the effective concentration of the enzyme. This blunts the effect of the statins and limits the extent of cholesterol reductions attainable.

The IPI and/or prenyl transferase inhibitors of the present invention do not act directly on HMGCoA reductase and so disrupt a smaller range of metabolite pools than the statins. They therefore have important benefits as hypolipidaemics. In particular, they exhibit reduced side effects, greater activity as hypolipidaemics and can be used in a broader class of patients.

The IPI and/or prenyl transferase inhibitors of the invention can be used as hypolipidaemic agents, and in particular as hypocholesterolaemic agents. They find application in lowering serum cholesterol levels, in the treatment or prophylaxis of hypercholesterolaemia, hyperlipidaemia, hyperlipoproteinaemia, nephrotic hyperlipidaemia or atherosclerosis, in increasing HDL cholesterol levels while lowering LDL cholesterol and serum triglyceride levels, in the treatment or prophylaxis of cardiovascular disease (e.g. atherosclerosis and other arterial lesions) and for the prevention of restenosis after coronary angioplasty.

The IPI and/or prenyl transferase inhibitors of the invention may be administered to any or all of the following classes of patients:

(a) hepatocompromized individuals;

(b) individuals with a prior history of liver dysfunction;

(c) individuals with 200-300 mg/dl serum cholesterol;

(d) mammals (e.g. humans);

(e) individuals lacking a functional LDL receptor; and

(f) individuals suffering from familial hypercholesterolaemia

(e.g. homozygous familial hypercholesterolaemia)

In particular, the IPI and/or prenyl transferase inhibitors of the invention may be used in the treatments of any of the following types of hyperlipoproteinaemias:

Type I (hyperchylomicronaemia) characterised by the presence of chylomicrons and by normal or only slightly increased concentrations of very low-density lipoproteins;

Type IIa (hyper-β-lipoproteinaemia) characterised by an elevation in the concentration of low-density lipoproteins;

Type IIb characterised by an elevation in the concentration of low-density lipoproteins and of is very low-density lipoproteins;

Type III (‘floating β’ or ‘broad β pattern) characterised by the presence of very low-density lipoproteins having an abnormally high cholesterol content and an abnormal electrophoretic mobility;

Type IV (hyperpre-β-lipoproteinaemia) characterised by an elevation in the concentration of very low-density lipoproteins, but no increase in the concentration of low-density lipoproteins, and by the absence of chylomicrons;

Type V (‘hyperpre-β-lipoproteinaemia and chylomicronaemia’) characterised by an elevation in the concentration of very low-density lipoproteins and the presence of chylomicrons.

They may also be used to treat primary hyperlipidaemias in any of the following categories:

(a) familial hypercholesterolaemia, which is usually heterozygous but very rarely may be homozygous, characterised by a type IIa pattern (occasionally a type IIb pattern is present );

(b) familial hypertriglyceridaemia, usually associated with a type IV or type V patter;

(c) familial combined hyperlipidaemia, characterised by elevated cholesterol only, elevated triglyceride only, or elevated cholesterol and triglyceride—type IIa, type IV, or type IIb patterns may be found;

(d) familial dysbetalipoproteinaemia (remnant hyperlipoproteinaemia or broad-β disease) showing the type III pattern; and

(e) lipoprotein lipase deficiency or apolipoprotein C-II deficiency showing a type I of type V pattern.

The IPI and/or prenyl transferase inhibitors of the invention also find application in the modulation of cell proliferation and in particular in the treatment of prophylaxis of disorders involving cell proliferation (for example various cancers, including cancers and cancer metastases in bone).

Products of the mevalonate pathway are critical for the proliferation of many cells. As upstream inhibitors, IPI and/or prenyl transferase inhibitors block the synthesis of these products, and are therefore also useful in the treatment of diseases characterised by cell proliferation.

The prenyl transferase and/or IPI inhibitors of the invention also potentiate the effects of other drugs and treatments affecting cellular growth, such as radiotherapy, surgery and drugs used in cancer chemotherapy, immunotherapy or immunosuppression. Thus, the IPI and/or prenyl transferase inhibitors of the present invention may be administered as adjuncts to radiotherapy, chemotherapy, immunotherapy or surgery in the treatment of cancer, particularly of ras-related cancers. Examples of ras-related cancers include lung, bladder, colon and brain cancers.

The activity of the IPI and/or prenyl transferase inhibitors of the invention in this respect contrasts with that of the protein-prenyl transferase inhibitors described for use in blocking neoplastic transformation in EP0537008. Protein-prenyl transferase acts at a point downstream of farnesyl pyrophosphate (see FIG. 1) and the inhibitors described in EP0537008 do not block or reduce the flow of farnesyl pyrophosphate into the various prenylation pathways. In this respect they differ fundamentally from the IPI and/or prenyl transferase inhibitors of the present invention.

The anti-cancer activity of the IPI and/or prenyl transferase inhibitors of the invention arise from a block of protein prenylation (for example, prenylation of CaaX box containing proteins such as the G proteins, members of the ras family and other oncoproteins) by reducing or eliminating input into the isoprenoid pathways shown in FIG. 1. This leads inter alia to a reduction or elimination of protein-membrane interaction necessary for the transduction of proliferation signals across membranes. Also, the consequent shrinkage of dolichol pools blocks the synthesis of glycoproteins and changes membrane fluidity, which are important for cell growth.

In particular, the IPI and/or prenyl transferase inhibitors of the present invention reduce metabolic pools of prenyls (e.g. farnesyl pyrophosphate) which are involved in the post-translational modification of CaaX box containing proteins (including proteins in the ras family and G-proteins). For example, the inhibitors of the present invention reduce metabolic pools of farnesyl pyrophosphate, which is the donor of the farnesyl group to CaaX box containing proteins such as the ras p21 oncoprotein.

Thus, the activity of CaaX containing proteins such as the ras oncoprotein and G proteins can be blocked by preventing their post-translational modification by administration of the inhibitors of the invention. In particular, the prenyl transferase and/or IPI inhibitors of the invention can be used to prevent prenylation (including geranylation, geranylgeranylation or farnesylation) of proteins (for example CaaX box containing proteins) and ras in particular. By blocking or reducing the post-translational modification of proteins (such as CaaX box containing proteins) the inhibitors of the present invention effectively abolish the transforming activity of these proteins. Thus, the inhibitors of the invention find application in the treatment and/or prophylaxis of tumours.

In preferred embodiments, the prenyl transferase and/or IPI inhibitors of the invention are used to prevent or reduce the farnesylation of ras in the modulation of cell proliferation, isoprenoid-related cellular apoptosis or cellular signal transduction.

The inhibitors of the present invention also find application in the modulation of isoprenoid-related cellular apoptosis, for example in the treatment or proyphlaxis of various autoimmune diseases (such as rheumatoid arthritis) and chronic inflammatory diseases.

The inhibitors of the present invention also find application in the modulation of cellular signal transduction, for example in the treatment or prophylaxis of graft (e.g. allograft) rejection. Protein prenylation increases protein hydrophobicity and promotes protein-membrane interactions. Prenylated ras protein localizes to the inner cell membrane and appears to function as a mediator through which various peptide growth factors and cytokines signal to stimulate intracellular events.

Binding of these substances to their respective receptors on target cells activates ras and triggers inter alia cell proliferation, differentiation, and T-cell activation.

As described earlier, the inhibitors of the invention block or reduce ras prenylation, and ras has now been discovered to play an important role in chronic allograft rejection (O'Donnell et al., 1995, Kidney International, Vol. 48, Suppl. 52, pp. S-29-S-33). Thus, by interfering with ras protein prenylation or activation by growth factor receptors the prenyl transferase inhibitors of the invention ameliorate chronic rejection.

When administered in suitable concentrations, the IPI and/or prenyl transferase inhibitors of the present invention can also be used to block sterol synthesis in plants and fungi. They therefore find utility as fungicides and/or herbicides.

Without wishing to be bound by any theory, the IPI and/or prenyl transferase inhibitors of the invention are thought to act as stable analogues of the carbocation intermediate shown in FIG. 2, thereby blocking the synthesis of farnesyl pyrophosphate.

Accordingly, the prenyl transferase inhibitors of the present invention preferably comprise structural and/or functional analogues of the carbocation:

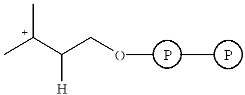

These analogues may be bisphosphonates (either geminal or non geminal bisphosphonates), and may be structural and/or functional analogues of the carbocation:

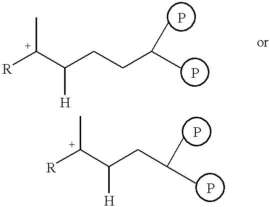

wherein R is an alkyl side chain, for example having >5 (for example about 10) carbon atoms.

The analogues may also be sulphonates, carboxylates, phosphonocarboxylates, phosphonosulphonates, phosphonophosphates, or pyrophosphates.

Preferred inhibitors constitute a subclass of bisphosphonates which inhibit either or both of IPI and prenyl transferase (the latter hereinafter referred to as prenyl transferase inhibitory bisphosphonates or PIBs and the former as IPI inhibitory bisphosphonates or (PIBs), or analogues or derivatives thereof. The IPIB/PIB is preferably selected from the large number of known osteoactive bisphosphonates (see e.g. Fleisch, H., 1993, Bisphosphonates in bone disease, ISBN 3-9520459-O-X, the contents of which are incorporated herein by reference). The IPI and/or prenyl transferase inhibitor of the invention may also be an analogue or derivative of the osteoactive IPIB or PIB, for example a derivative having a group for promoting cellular uptake (e.g. a lipophilic group or a dipeptide group).

The term “lipophilic group” as used herein encompasses a group containing at least about 6 carbon atoms (preferably at least 10) and less than about two (preferably no more than five) polar substituents bearing OH, NH or C═C functions.

Preferably, the IPI and/or prenyl transferase inhibitors have a positively charged nitrogen atom. Particularly preferred are PIBs or IPIBs having a positively charged nitrogen atom. Examples of such bisphosphonates are alendronate, pamidronate and ibandronate, and analogues or derivatives thereof including nitrogen ring-containing (heterocyclic) compounds.

The IPI prenyl transferase inhibitors may be modified in a variety of ways to modify their pharmacokinetics and in vivo localization. When used as hypolipidaemics, the IPI/prenyl transferase inhibitors may be modified or derivatized to render them more lipophilic using techniques known to those skilled in the art. For example, the inhibitors may be modified by the attachment of a lipophilic group or of a dipeptide group permitting uptake via the cellular plasma membrane peptide carrier system (see e.g. work by Breuer et al at the School of Pharmacy at the Hebrew University of Jerusalem). Thus, the inhibitors of the invention may be prodrugs having enhanced bioavailablity.

When used to treat cancers, the prenyl transferase/IPI inhibitors may be modified to target them to the tumour site. The IPIBs/PIBs accumulate in bone, and so are particularly preferred in the treatment-of cancers and cancer metastases in bone.

Prenyl Transferase and/or IPI Inhibitors and Bone Disease

The invention also relates to the use of an IPI and/or prenyl transferase inhibitor for the manufacture of a medicament for the regulation of bone metabolism, for example in the treatment of Paget's disease, hypercalcaemia (both tumour-induced and non-tumour induced), bone metastases or osteoporosis in both men and women (e.g. associated with secondary causes such as administration of glucocorticoids), wherein the inhibitor is not a geminal bisphosphonate.

The invention permits the identification of improved phosphonate drugs with reduced affinity for bone.

In another aspect, the invention relates to an IPI and/or prenyl transferase inhibitor for use in therapy or prophylaxis, e.g. in the manufacture of a medicament for the regulation of bone is metabolism, for example in the treatment of Paget's disease, hypercalcaemia (both tumour-induced and non-tumour induced), bone metastases or osteoporosis, wherein the inhibitor is not a geminal bisphosphonate.

The term bisphosphonate is used herein in a broad sense to cover not only bisphosphonate sensu stricto, but also bisphosphonate analogues Bisphosphonate analogues are those ligands which can compete with osteoactive bisphosphonate for binding to the IPI and/or prenyl transferase of the invention, or which can compete in vitro (for example, on an affinity column) with osteoactive bisphosphonate (in either the free state or in the form of a derivative linked to an affinity column) for binding to the IPI and/or prenyl transferase of the invention.

Bisphosphonate analogues include pyridoxal phosphate, O-phosphorylethanolamine, o-phosphorylcholine, phosphatidyl ethanolamine and phospholipid bisphosphonate analogues.

Osteoactive drugs (e.g. bisphosphonates) are those which act as antiresorptive and/or anti-inflammatory drugs in vivo.

The prenyl transferase and/or IPI inhibitor for use in the therapies listed above is preferably a structural and/or functional analogue of the carbocation:

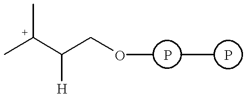

These analogues may be bisphosphonates (either geminal or non geminal bisphosphonates), and may be structural and/or functional analogues of the carbocation:

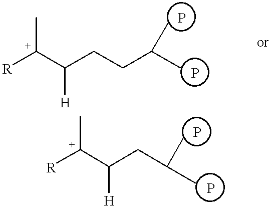

wherein R is an alkyl side chain, for example having >5 (for example about 10) carbon atoms.

The inhibitors may also be sulphonates, carboxylates, phosphonocarboxylates, phosphonosulphonates, phosphonophosphates, or pyrophosphates.

The IPI and/or prenyl transferase inhibitor may comprise a positively charged nitrogen atom.

The invention also contemplates the use of IPI and/or prenyl transferase for binding a bisphosphonate, for example in an in vitro assay.

Preferably, the IPI and/or prenyl transferase is one which, in vivo, mediates the physiological (e.g. antiresorptive) effects of osteoactive bisphosphonate. It may also be a Dictyostelium spp., for example Dictyostelium discoideum, prenyl transferase.

The invention also embraces a method for screening for osteoactive drugs (e.g. bisphosphonates) comprising the steps of: (a) contacting a putative drug (e.g. a bisphosphonate) with the IPI and/or prenyl transferase of the invention, and (b) determining whether interaction (e.g. binding) between the putative drug and IPI and/or prenyl transferase occurs, wherein interaction is indicative of an osteoactive drug (e.g. an osteoactive bisphosphonate). The relative affinity (interaction) with IPI and prenyl transferase may also be determined in the methods of the invention.

Conveniently, the presence or degree of interaction between the putative drug and IPI and/or prenyl transferase is measured or determined by an enzyme inhibition assay. Such assays may be conducted according to any of the standard IPI and/or prenyl transferase assay protocols known to those skilled in the art.

The method described above is useful for screening large numbers of synthetic drugs for therapeutic activity. It may also be used to classify potential osteoactive or anti-arthritic bisphosphonates into different groups according to their mode of action (or their cellular targets). Preferably, the method is employed in high throughput screening of drug candidates. Compounds identified by the method of the invention can be further modified or used directly as therapeutic compounds, for example in the treatment of osteoporosis.

Also contemplated is a method for evaluating the therapeutic activity of a putative drug (e.g. a bisphosphonate) comprising the steps of: Ca) contacting the drug with IPI and/or prenyl transferase, and (b) measuring the binding affinity of the putative drug for the prenyl transferase and/or IPI, or the relative binding affinities for prenyl transferase and IPI.

The method described above is useful e.g. for ranking the therapeutic activity of potential drugs (e.g. potential bisphosphonate drugs).

In another aspect the invention relates to a method for synthesising a therapeutically active (e.g. antiresorptive or antiarthritic) drug (e.g. a bisphosphonate) comprising the steps of: (a) providing a three-dimensional model comprising a catalytic site of the prenyl transferase of the invention (e.g. by computer analysis of its amino-acid sequence or by X-ray crystallography of the prenyl transferase or fragment thereof), and (b) modelling the therapeutically active drug with reference to the three-dimensional model generated in step (a).

Many different techniques exist for generating a three-dimensional model for use in the above-described method, and all are suitable for use in the method of the invention. Conveniently, the three-dimensional model is generated by computer analysis of the amino-acid sequence of all or a portion of the IPI prenyl transferase (for example a catalytic site thereof or a bisphosphonate binding domain thereof). Examples of such models are known to those skilled in the art.

Alternatively, the three-dimensional model could be generated by X-ray crystallography of prenyl transferase/IPI (or fragments/derivatives thereof), or by NMR techniques. These techniques could also be applied to the IPI/prenyl transferase-drug (e.g. IPI/prenyl transferase-bisphosphonate) complex, the results of which could also be used as the basis for the rational design of therapeutic agents.

The invention also covers therapeutically active drugs (e.g. a bisphosphonate) which have been screened, evaluated or synthesised by the methods of the invention.

The invention also contemplates an antibody (e.g. a monoclonal antibody) which binds to the IPI and/or prenyl transferase of the invention.

The antibodies of the invention may advantageously bind specifically to the IPI and/or prenyl transferase of the invention (e.g. specifically to both). Antibodies specific for the catalytic site of IPI and/or prenyl transferase may act as bisphosphonate mimetics. Specific binding may be exploited in imaging techniques, for example to assess the extent to which osteoactive drug targets (e.g. bisphosphonate targets) are available for drug action, or to determine the degree of occupancy of drug targets in patients undergoing therapy.

The invention also contemplates antibody derivatives, including antibody fragments (e.g. Fab fragments), chimaeric antibodies (including humanized antibodies) and antibody derivatives (such as fusion derivatives comprising an antibody-derived variable region and a non-immunoglobulin peptide having for example enzyme or conjugative activity).

The invention also covers test kits comprising the IPI and/or prenyl transferase of the invention, for example for use in the screening or evaluation methods of the invention. In such test kits: (i) the IPI and/or prenyl transferase may be bound to a solid support and/or (ii) the kit further may comprise a labelled (e.g. radiolabelled or fluorescently labelled) bisphosphonate and/or (iii) the kit may further comprise the antibody of the invention.

The invention also covers the individual components of such kits per se. In particular, the invention contemplates the IPI prenyl transferase of the invention when bound to a solid support.

Also contemplated by the invention is a mimetic or antagonist of the IPI prenyl transferase of the invention, the mimetic for example consisting essentially of the catalytic or bisphosphonate binding site of IPI or prenyl transferase.

The invention also contemplates various therapeutic applications for the antibody, mimetic or antagonist of the invention.

In another aspect, the invention relates to a process for producing an osteoactive drug comprising the step of providing an analogue of the carbocation:

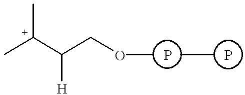

which analogue has a positively charged nitrogen atom in an alkyl chain thereof.

In a further aspect, the invention relates to a process for selectively inhibiting an enzyme involved in sterol/isoprenoid biosynthesis comprising the stem of providing an analogue of the carbonation:

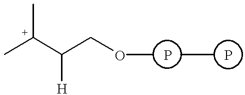

which analogue has a positively charged nitrogen atom in an alkyl chain thereof.

The carbocation preferably is:

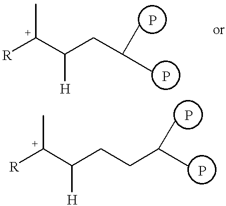

wherein R is an alkyl side chain, for example having >5 (for example about 10 or about 15) carbon atoms.

Particularly preferred analogues are bisphosphonates having a formula selected from:

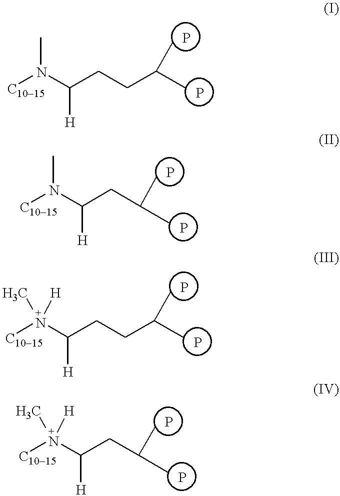

The enzyme to be selectively inhibited is preferably selected from the enzymes squalene synthase, protein prenyl transferase, cis-prenyl transferase and trans-prenyl transferase (geranylgeranylpyrophosphate synthase).

The selectivity of inhibition need not be absolute, and the compounds of the invention may inhibit two or more of the above-listed enzymes. The spectrum of inhibitory activity over the enzymes listed above (i.e. the relative or absolute inhibitory specificity) may be selected by varying inter alia the length of the carbon side chain (shown as C₁₀₋₁₅ above). For example, greater inhibitory selectivity can be achieved by providing a long (e.g. about C₁₅ or longer) carbon side chain.

The invention therefore also contemplates a method of varying the inhibitory profile of an inhibitor of enzyme(s) involved in sterol/isoprenoid biosynthesis by varying the length of the carbon side chain shown in the general formula below:

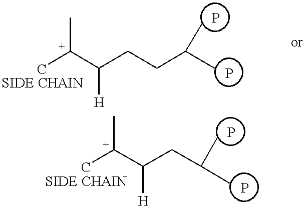

Particularly preferred are geranylgeranylpyrophosphate synthase inhibitors. Such inhibitors preferably have the

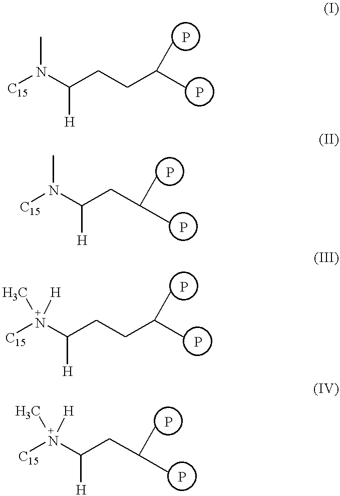

Drugs based on or derived from such inhibitors would allow specific inhibition of prenylation of proteins that are modified by a geranylgeranyl group without inhibiting sterol biosynthesis or protein farnesylation.

In such processes, the drug or inhibitor may not be a bisphosphonate. Alternatively, the drug or inhibitor may be a bisphosphonate (for example a geminal or non geminal bisphosphonate), sulphonate, carboxylate, phosphonocarboxylate, phosphonosulphonate, phosphonophosphate or pyrophosphate.

The drug or inhibitor may be further modified to facilitate cellular uptake, for example by the attachment of a lipophilic group or by the attachment of a dipeptide group permitting uptake via the cellular plasma membrane peptide carrier system.

In another aspect, the invention relates to a process for increasing the therapeutic efficacy of an osteoactive bisphosphonate comprising the step of introducing a positively charged nitrogen atom into an alkyl chain thereof.

The IPI and/or prenyl transferase inhibitors of the invention can be administered in a variety of forms, including oral and parental routes. Parental administration can be intravenous, intramuscular, subcutaneous, intrasynovial, transdermal, intraocular, sublingual, buccal, topically or rectal. Other routes include nasal inhalation e.g. via a nebulizer, atomizer or aerosol.

The IPI and/or prenyl transferase inhibitors may be formulated in an inert diluent or with an assimilable edible carrier. It may be enclosed in hard or soft shell capsules or compressed into tablets. For oral therapeutic administration, the active compound may be incorporated with excipient and used in the form of ingestible tablets, buccal tablets, troches, capsules, elixirs, suspensions, syrups or wafers. Preferred compositions or preparations according to the present invention are prepared so that an oral dosage unit form contains between abort 10 and about 1000 mg (e.g. between 50 and 300 mg) of active compound.

The tablets, troches, pills, capsules and the like may also contain a binder (for example corn starch or gelatin), excipients such as dicalcium phosphate, disintegrating agents, lubricants and sweetening or flavouring agents. When the dosage unit form is a capsule, it may contain, in addition to materials of the above type, a liquid carrier. Unit dosage forms are pharmaceutically pure. The inhibitors may be incorporated into sustained-release preparations and formulations.

The active compound may also be administered parenterally or intraperitoneally. Solutions or suspensions of the a active compounds or pharmacologically acceptable salt can be prepared in water suitably mixed with a surfactant. Dispersion can also be prepared in glycerol, liquid polyethylene glycols, mixtures thereof and oils.

The pharmaceutical forms suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), suitable mixtures thereof, and vegetable oils.

The dosage of the inhibitors is determined by the physician and varies with the form of administration, the active compound selected and the patient under treatment. The therapeutic dosage will generally be from about 0.1 to about 100 mg/kg/dy and may be administered in several different unit dosage form. Higher dosages may be required for oral administration.

### EXAMPLE 1

### Inhibition of Farnesyl Diphosphate Synthase (Prenyl Transferase) and IPI by Bisphosphonates

| | Prenyl transferase IC₅₀(nM)* | IPI |
|---|---|---|
| Bisphosphonate | (FPP synthase) | (IPP isomerase) |
| PAMIDRONATE | 5000 | 200 |
| ALENDRONATE | 2000 | 75 |
| YM175 | 45 | 35 |
| IBANDRONATE | 20 | 35 |
| RISEDRONATE | 20 | 22 |

*Concentration required to inhibit enzyme activity by 50%

### Methods

Chemicals

Clodronate, alendronate, ibandronate, YM175 and risedronate were provided by Procter and Gamble Pharmaceuticals, Cincinnati, Ohio. The bisphosphonates were dissolved in PBS, the pH adjusted to 7.4 with 1N NaOH, then filter-sterilised by using a 0.2 μm filter. Mevastatin (also known as compacting was purchased from Sigma Chemical Co, Poole, UK, and converted from the lactone form by dissolving 5 mg mevastatin in 100 μl 1N NaOh. After addition of 1 ml PBS, the pH of the solution was adjusted to approximately pH 8 using 1N HCl, then filter-sterilised. A stock solution of 10 mM mevalonic acid lactone was prepared by dissolving the solid in dry ethanol, while farnesylpyrophosphate (FPP) and geranylgeranylpyrophosphate (GGPP), purchased from Sigma, were dried to remove solvent then resuspended in culture medium immediately before use. Methionine and mevalonolactone was from Amersham, Aylesbury, UK. All other chemicals were from Sigma Chemical Co, Poole, UK, unless stated otherwise.

Cell Culture

J774.2 cells were obtained from the European Collection of Animal Cell Cultures (Porton Down, UK). Cultures were grown at 37° C. in Dulbecco's Modified Eagle Medium (GIBCO, Paisley, UK) containing 10% heat inactivated foetal calf serum, 100 μg/ml penicillin, 100 μg/ml streptomycin and 1 mM glutamine in 5% CO₂ atmosphere.

Assessment of Apoptosis in J774 Macrophages

Apoptosis in J774 macrophages was identified on the basis of characteristic changes in nuclear morphology i.e. condensation of chromatin and fragmentation of nuclei into apoptotic bodies, that distinguish apoptotic cells from cells undergoing necrotic cell death.

J774 cells were seeded into 12 well plates (Costar) at a density of 10⁵ per well. After 24 h, the medium was replaced with fresh medium containing either 1-100 μM mevastatin, 100 μM alendronate or 15 μM mevastatin, with or without 0.5 μM cycloheximide, 200 μM FPP, 200 μM GGPP or 200 μM mevalonic acid lactone. After 48 h, both adherent and non-adherent cells were collected, fixed with 4% (v/v) formaldehyde than cytospun onto slides and visualised as described by Rogers et al. In addition, DNA was extracted from approximately 5×10⁵ J774 cells following treatment with 20 μM or 100 μM mevastatin for 48 h, and analysed for the presence of oligonucleosome-sized fragments by agarose gel electrophoresis.

The effect of cycloheximide, FPP, GGPP and mevalonic acid lactone on loss of total cell viability owing to apoptosis was also assessed by measuring the ability of cells to metabolise MTT (3-(4,5-dimethylthiazol-2yl)-2,5-diphenyltetrazolium bromide). 96-well plates (Costar) were inoculated with 1.5×10⁴ cells per well. 24 h later the cells were treated with 15 μM mevastatin or 100 μM alendronate, together with 0.5 μM cycloheximide, 200 μM FPP, 200 μM GGPP or 200 μM mevalonic acid lactone. After 48 h incubation, the conversation of MTT reagent by viable cells was measured spectrophotometrically according to Rogers et al (1996).

Measurements of Protein Prenylation in J774 Macrophages

The ability of bisphosphonates to affect protein prenylation in J774 cells was investigated by measuring incorporation of mevalonate into proteins post-translationally modified with farnesyl and geranylgeranyl groups. Cells were seeded into 6 well plates at a density of 5×10⁵ cells per well. After 24 h, the medium was replaced and cells were starved for mevalonate by incubation with 5 μM (final concentration) mevastatin for 4 h. The medium was then replaced with 1.0 ml fresh medium containing 7.5 μCi/ml mevalonate (specific activity 57 mCi/mmol) and either 0.1 μM bisphosphonate or vehicle (PBS). After 24 h (the approximate time at which apoptotic cells appear in bisphosphonate-treated cultures) the adherent and non-adherent cells were collected and centrifuged at 1000 g for 5 min. The cells were then lysed in 0.5 ml RIPA buffer (PBS, 0.1% (w/v) sodium dodecyl sulfate, 0.5% (w/v) sodium deoxycholate, 10 82 g/ml phenylmethylsulfonyl fluoride). The protein concentration of the lysates was determined using the BCA protein assay (Pierce). Equal quantities of protein (usually 50 μg) of each lysate were then electrophoresed on 12% polyacrylamide-SDS gels under reducing conditions. After electrophoresis, the gels were dried then visualised after exposed to a high sensitivity phosphoimaging screen (BioRad) for 3 days.

Immunoprecipitation of Ras and Rab6 from J774 macrophages

Approximately 10⁷ cells in 75 cm² flasks were treated for 16 h with 100 μM alendronate or 15 μM mevastatin. Control cells were treated with vehicle (PBS). The cells were then incubated with 8 ml methionine-free medium (containing 100 μM alendronate or 15 μM mevastatin) for 1 h, before addition of 120 μCi methionine (specific activity 1000 C1/mmol) and further incubation for 24 h. Adherent and non-adherent cells from each flask were then harvested and lysed in 1.0 ml of RIPA buffer. Lysates for immunoprecipitation of Rab6 were precleared by using 1 μg of rabbit IgG and 20 μl protein A agarose slurry, followed by addition of 2 μg polyclonal rabbit anti-Rab6 (santa Cruz) for 2 h, then 50 μl protein A-agarose and incubation overnight. Ras was immunoprecipitated by overnight incubation, at 4°, of 1 ml lysate with 30 μl pan-Ras antibody. Y13-259 conjugated to agarose beads (Oncogene Science). Immunoprecipitates were washed 5 times with 1 ml RIPA buffer, then bound proteins were removed by boiling for 4 minutes in 30 μl Laemmli sample buffer. Finally, samples were electrophoresed on 12.5% polyacrylamide-SDS gels under reducing conditions and detected as described above.

### Results

An Inhibitor of the Mevalonate Pathway Caused Macrophage Apoptosis

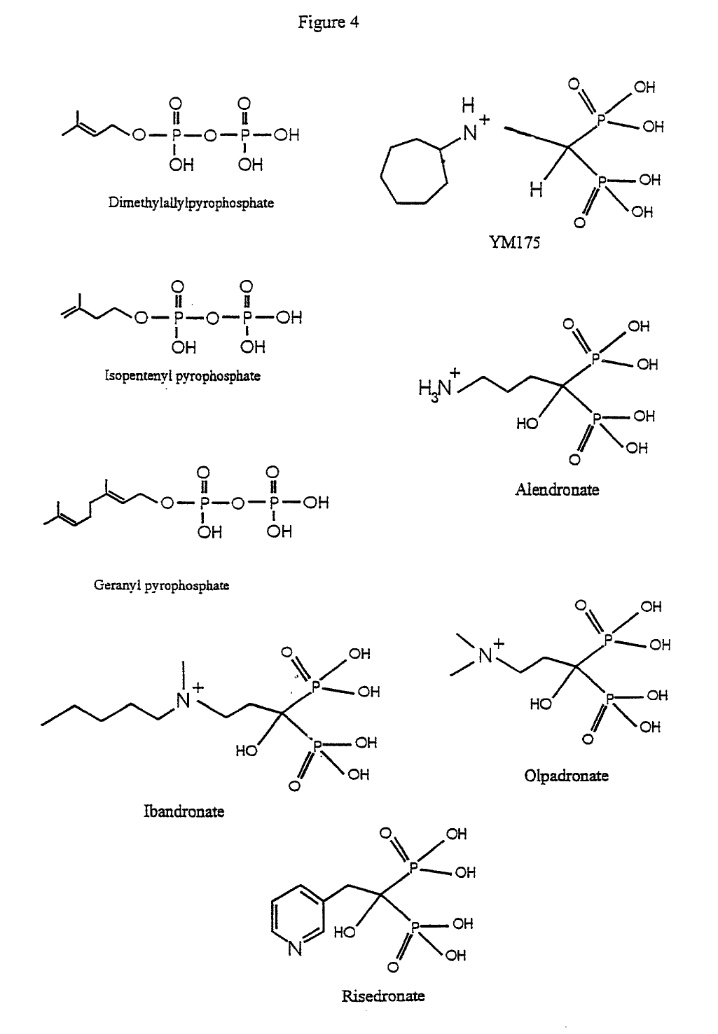

Concentrations of 10-100 μM mevastatin, an inhibitor of HMGCoA reductase (which catalyses the synthesis of mevalonate, FIG. 1) caused a dose-dependent increase in the proportion of J774 cells with the morphological and biochemical features typical of apoptosis i.e. chromatin condensation and formation of apoptotic bodies (FIG. 2) and oligonucleosomal DNA fragmentation (FIG. 3). Mevastatin appeared to be more potent at inducing apoptosis than alendronate or risedronate, since concentrations of approximately 10 μM mevastatin, 30 μM alendronate or 3 μM risedronate caused 50% loss of total cell viability after 48 h (FIG. 4).

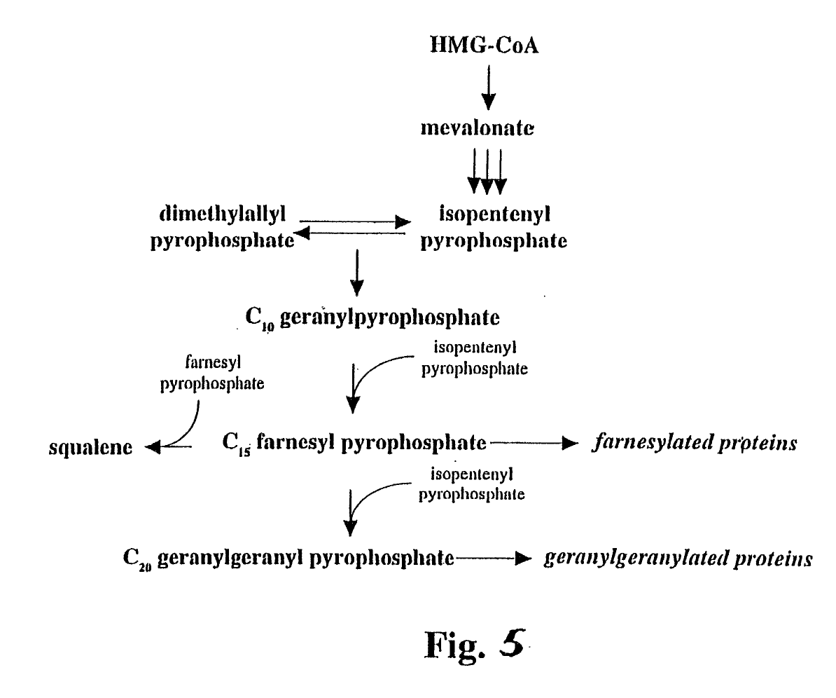

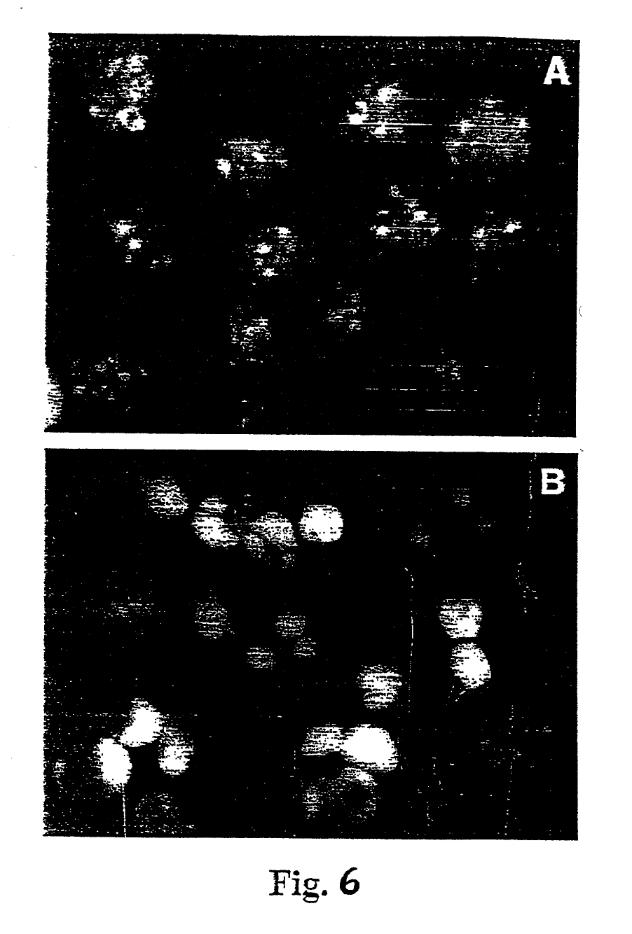

Mevastatin-induced loss of cell viability, measured by reduction of MTT reagent, could be prevented by co-incubating J774 cells with 15 μM mevastatin and 0.5 μM cycloheximide during the 48 h culture period (FIG. 5). Analysis of the proportion of morphologically apoptotic cells also demonstrated that cycloheximide prevented apoptosis (not shown). Co-incubation with 200 μM FPP, GGPP or especially mevalonic acid lactone also prevented (at least partially) mevastatin-induced loss of cell viability and apoptosis (FIG. 6). By contrast, apoptosis and loss of cell viability caused by 100 μM alendronate could be partially inhibited only by co-incubation with 200 μM FPP or GGPP but not with 200 μM mevalonic acid lactone (FIG. 6).

Bisphosphonates Inhibit Post-translational Prenylation of Proteins

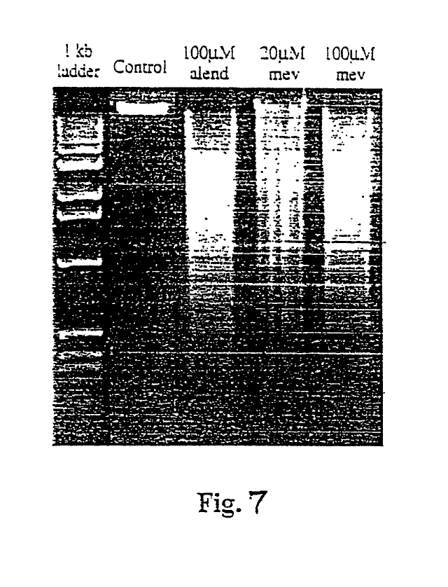

J774 cells metabolically-labelled with mevalonolactone for 24 h contained radiolabelled proteins that could be separated by electrophoresis on 12% polyacrylamide gel into proteins of molecular weight 21-26 kDa (mostly geranygeranylated GTP-binding proteins, but also farnesylated Ras proteins), 60-70 kDa (farnesylated lamin B and prelamin A), 17 kDa and 46 kDa. A broad band at the migrating front of the gels (which did not stain with Coomassie blue and was not affected by prior treatment of cell lysates with proteinase K or RNase¹⁴) was most likely radiolabelled, pyrophosphate-containing intermediates of the mevalonate pathway, such as FPP and GGPP. Treatment of J774 cells with 100 μM alendronate, ibandronate and risedronate during the 24 h labelling period markedly reduced the incorporation of radiolabel into all the protein bands (but especially 21-26 kDa, mostly geranylgeranylated, proteins) and reduced the amount of radiolabelled compounds at the dye front (FIG. 7). 100 μM YM175 inhibited even more effectively the incorporation of radiolabel into proteins and into compounds at the dye front. By contrast, 750 μM clodronate (a concentration that causes a substantial reduction in viability of 3774 cells) did not affect protein prenylation or synthesis of the radiolabelled dye-front compounds. Hence, there was some correlation between the ability of the bisphosphonates to inhibit protein prenylation in J774 cells and the anti-resorptive potency of the bisphosphonates (risedronate>YM175>i bandronate>alendronate>>clodronate). Inhibition of protein prenylation was not the result of an inhibitory effect of the bisphosphonates on de novo protein synthesis, since 24 h treatment with 100 μM of the bisphosphonates does not inhibit incorporation of methionine into protein in J774 cells.

Bisphosphonates Inhibit Farnesylation of Ras and Geranylgeranylation of Rab6

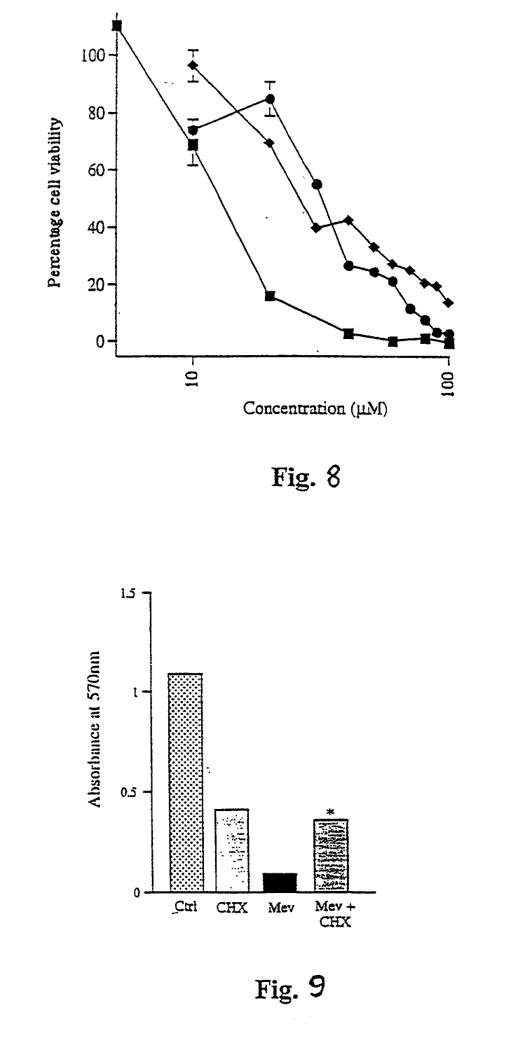

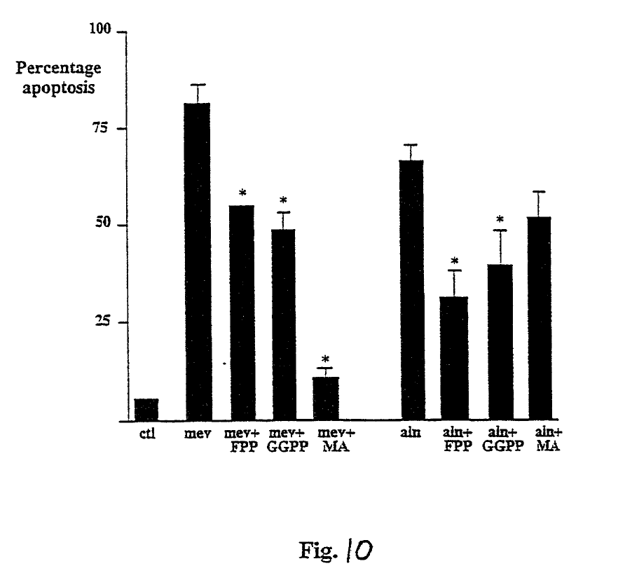

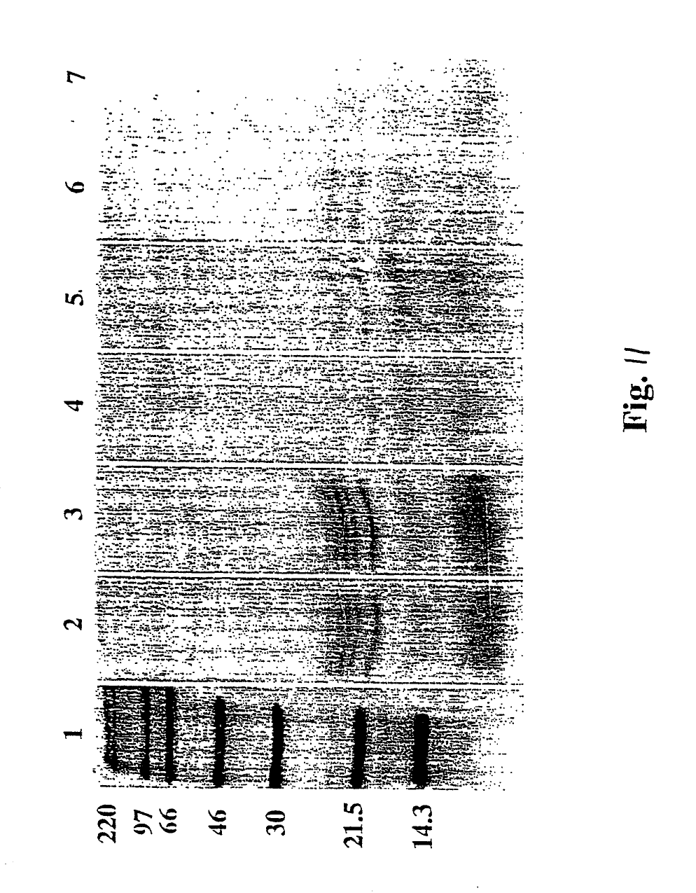

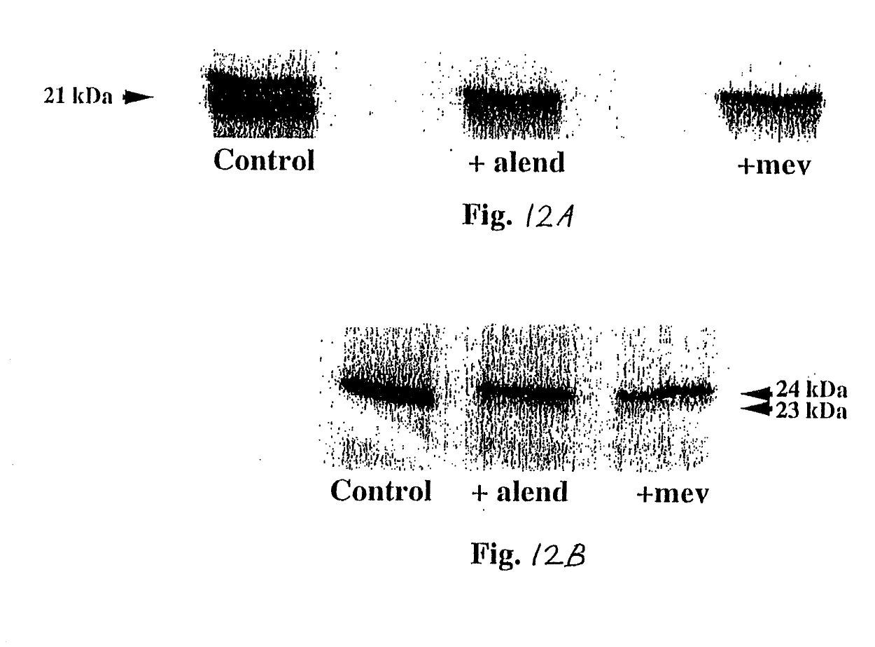

To demonstrate further that both farnesylated and geranylgeranylated proteins were affected by bisphosphonates, we immunoprecipitated Ras and Rab6 proteins from cell lysates of J774 macrophages that had been metabolically labelled with methionine. Immunoprecipitation of Ras from cell lysates of control J774 cells, using the pan-Ras antibody Y13-259, gave rise to two bands of around 21 kD following electrophoresis of immunoprecipitates on 12.5% polyacrylamide gels. These comprised the non-farnesylated form of Ras (the upper band) and farnesylated Ras (the lower band, which migrates faster owing to removal of the terminal tripeptide following prenylation). After treatment of J774 cells for 41 h with 100 μM alendronate (by which time about 80% of the remaining cells were apoptotic) then immunoprecipitation of Ras with the Y13 antibody, the non-farnesylated form was predominant and the farnesylated form became barely detectable (FIG. 8A). Identical results were obtained after treatment with 15 μM mevastatin for 41 h.

Alendronate and mevastatin also prevented geranylgeranylation of Rab6. Immunoprecipitation of Rab6 from lysates of J774 cells that had been treated with 10 μM alendronate or 11 μM mevastatin for 41 h gave rise to a prominent band of 24 kD, non-prenylated Rab6 and a barely detectable 23 kD geranylgeranylated form (FIG. 8B). In lysates from control cells, Rab6 was immunoprecipitated entirely as the geranygeranylated form.

### Discussion

Our observations demonstrate that potent anti-resorptive bisphosphonate drugs such as risedronate, YM175, ibandronate and alendronate can inhibit post-translational modification of proteins with isoprenoid (farnesyl or geranylgeranyl) groups. In 774 macrophages, the incorporation of mevalonate into prenylated proteins, including lamins, Ras and Rab6, was inhibited by a concentration of the bisphosphonates that also causes apoptosis in vitro (100 μM). Furthermore, another inhibitor of protein prenylation, mevastatin, was even more potent than alendronate at causing macrophage apoptosis. Hence, inhibition of protein prenylation is a likely route by which bisphosphonates cause apoptosis in J774 macrophages. The fact that clodronate did not inhibit the incorporation of mevalonate into prenylated proteins, does not inhibit sterol biosynthesis in vitro and is much less potent at causing 3774 apoptosis also supports the view that this bisphosphonate affects cells by a mechanism which is different to that of the more potent bisphosphonates such as risedronate and alendronate.

Induction of J774 apoptosis by bisphosphonates and by mevastatin could result from incorrect assembly of the nuclear lamina following loss of prenylation of lamin proteins, thus allowing endonucleolytic digestion of chromatin. Accumulation of non-prenylated GTO-binding proteins such as Ras could also lead to apoptosis, possibly as a result of intracellular acidification following loss of Ras- or Rho-dependent pH homeostasis. Indeed, we have found that bisphosphonate-induced and mevastatin-induced apoptosis in J774 macrophages is associated with cytoplasmic acidification (M. J. Rogers unpublished observations). In addition, we have previously reported that bisphosphonate-induced apoptosis in J774 cells can be prevented by actinomycin D and cycloheximide. Mevastatin-induced apoptosis in 3774 cells, like lovastatin-induced apoptosis in HL-60 cells, could also be prevented by cycloheximide. Since lack of prenylation of Ras, resulting in the accumulation of inactive cytoplasmic Ras, exerts a dominant negative effect on Ras signalling, one explanation for the protective effect of cycloheximide and actinomycin D against bisphosphonate- and mevastatin-induced apoptosis may be that cycloheximide and actinomycin D prevent Ras transcription and hence prevent accumulation of non-prenylated, cytoplasmic Ras.

The exact enzymes of the mevalonate pathway that are inhibited by bisphosphonates remain to be identified. Mevastatin is an inhibitor of HMG-COA reductase and thus prevents synthesis of mevalonate. Hence, mevastatin-induced apoptosis could be prevented by addition of mevalonic acid lactone or the mevalonate-derived compounds FPP and GGPP. By contrast, addition of either FPP or GGPP, but not mevalonic acid lactone, partially prevented alendronate-induced apoptosis. Hence, alendronate appears to inhibit enzymes later in the mevalonate pathway than HMG-CoA reductase. It is possible that both FPP synthase and GGPP synthase are inhibited, since bisphosphonates appeared to prevent the incorporation of mevalonate into FPP and GGPP. However, since FPP is itself converted to GGPP by GGPP synthase, it is also possible that the inhibitory effects of bisphosphonates on both farnesylation and geranylgeranylation of proteins could be the result of inhibition of FPP synthase alone. Alternatively, BPs could prevent the transfer of prenyl groups to proteins by prenyl protein transferases. It is perhaps more likely that bisphosphonates (like α-hydroxyfarnesylphosphonate) can actually inhibit several enzymes of the mevalonate pathway that contain similar prenyl-pyrophosphate binding sites. This is supported by the findings of Amin et al, since YM175 and ibandronate (but not alendronate) were potent inhibitors of squalene synthase, whilst we have demonstrated that YM175 and ibandronate probably also inhibit enzymes involved in FPP and GGPP synthesis. The potency of bisphosphonates could therefore depend on the combination of enzymes that are inhibited.

Since we have previously reported evidence that bisphosphonate-induced apoptosis in macrophages and osteoclasts probably occurs by the same mechanism, it is likely that potent bisphosphonates also induce osteoclast apoptosis owing to inhibition of protein prenylation. All the characteristic features of bisphosphonate-affected osteoclasts, including cytoskeletal rearrangement and loss of ruffled borders, decreased lysomal enzyme production and induction of apoptosis, can be accounted for as a result of loss of function of prenylated proteins such as Rho, Rab, Ras and lamins. The family of Rho and Rac proteins, for example, play an important role in regulating cytoskeletal organisation and cell morphology, including membrane ruffling, and whose function is essential for bone resorption by osteoclasts. Loss of function of Rab proteins could affect intracellular membrane trafficking and vesicular transport from the Golgi, and could therefore prevent the insertion of proton pumps into the osteoclast ruffled border. Induction of osteoclast apoptosis could result from effects on lamins or on Ras signalling, as discussed above. It is most likely that inhibition of bone resorption in vivo is actually a consequence of lack of prenylation of a multitude of osteoclast proteins. As in other cells, the extent to which prenylation is inhibited in osteoclasts (a reflection of the concentration of bisphosphonate to which osteoclasts are exposed and the length of exposure) may determine whether the cells simply lose the ability to function normally (i.e. fail to resorb bone) or whether this leads to induction of apoptotic cell death.

Several studies have suggested that bisphosphonates affect osteoclasts and other cells by interfering with cellular metabolism. Whilst protein tyrosine phosphatases have been postulated to be the molecular targets for alendronate, we have not found any increase in tyrosine phosphorylation in J774 macrophages undergoing apoptosis; inhibition of tyrosine phosphorylation in J774 cells by orthovanadate actually prevents bisphosphonate-induced apoptosis. Furthermore, inhibition of signal transduction processes by direct inhibition of tyrosine phosphatases would be expected to have rapid effects of osteoclasts. However, there are several reports that inhibition of osteoclastic resorption, like induction of osteoclast and J774 apoptosis, occurs many hours after first exposure to bisphosphonates, suggesting that bisphosphonates affect cell metabolism by an indirect effect. Furthermore, our observations that bisphosphonate-induced apoptosis could be partially prevented in the presence of FPP or GGPP clearly suggest that bisphosphonates induce apoptosis as a result of effects on the mavalonate pathway.

Given that protein prenylation is essential for all eukaryotic cells, inhibition of protein prenylation probably accounts for the effects of bisphosphonates on viability, growth and differentiation that have been observed with other mammalian cells, including osteoblast-like cells, myeloma cells, osteoclast precursors and even non-mammalian cells such as Dictyostelium amoebae. Identification of the inhibitory effect of bisphosphonates on protein prenylation has therefore finally shed new light on the mechanism of action of this hugely important class of anti-resorptive drugs.

1. Use of an inhibitor of IPI and/or prenyl transferase for the manufacture of a medicament for the modulation of lipid metabolism.

2. Use according to claim 1 wherein the medicament is a hypolipidaemic agent.

3. The use of claim 1 or claim 2 wherein the lipid is cholesterol.

4. The use of any one of claims 1 to 3 wherein the modulation:
(a) lowers serum cholesterol levels; and/or
(b) is in the treatment or prophylaxis of hypercholesterolaemia, hyperproteinaemia, hyperlipidaemia, hyperlipoproteinaemia, nephrotic hyperlipidaemia or atherosclerosis; and/or
(c) increases HDL cholesterol levels while lowering LDL cholesterol and serum triglyceride levels; and/or
(d) is in the treatment or prophylaxis of cardiovascular disease (e.g. arterial lesions); and/or
(e) is in the prevention of restenosis after coronary angioplasty.

5. The use of any one of claims 1 to 4 wherein the modulation is in:
(a) hepatocompromized individuals;
(b) individuals with a prior history of liver dysfunction;
(c) individuals with 200-300 mg/dl cholesterol;
(d) mammals (e.g. humans);
(e) individuals lacking a functional LDL receptor;
(f) individuals suffering from familial hypercholesterolaemia (e.g. homozygous familial hypercholesterolaemia).

6. Use of an innibitor of IPI and/or prenyl transferase for the manufacture of a medicament for the modulation of cell proliferation, for example by preventing or reducing the prenylation (e.g. farnesylation) of proteins (for example CaaX box-containing proteins, e.g. ras).

7. The use of claim 6 wherein the medicament is for the treatment or prophylaxis of a disorder involving cell proliferation.

8. The use of claim 7 wherein the disorder is cancer (e.g. cancers and cancer metastases in bone), for example a ras-related cancer (e.g. lung, bladder, colon or brain cancers).

9. Use of an inhibitor of IPI and/or prenyl transferase for the manufacture of a medicament for the modulation of isoprenoid-related cellular apoptosis, e.g. in the treatment of autoimmune disease (e.g. arthritis and inflammatory disease).

10. Use of an inhibitor of IPI and/or prenyl transferase for the manufacture of a medicament for the modulation of cellular signal transduction.

11. The use of claim 10 wherein the medicament modulates the ras protein.

12. The use of claim 10 or claim 11 wherein the modulation of cellular signal transduction is in the treatment of prophylaxis of graft (e.g. allograft) rejection.

13. A herbicide or fungicide comprising an IPI and/or prenyl transferase inhibitor.

14. Use of an inhibitor of IPI and/or prenyl transferase as a herbicide or fungicide.

15. The use according to any one of the preceding claims wherein the inhibitor is an analogue of the carbocation:

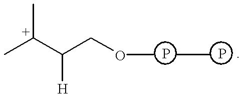

16. The use according to any one of the preceding claims wherein the inhibitor is a bisphosphonate, for example a geminal or non geminal bisphosphonate.

17. The use according to any one of claims 1 to 15 wherein the inhibitor is a sulphonate, carboxyl ate, phosphonocarboxylate, phosphonosulphonate, phosphonophosphate or pyrophosphate.

18. The use according to any one of the preceding claims wherein the inhibitor is an IPIB and/or PIB, or an analogue or derivative thereof.

19. The use according to claim 18 wherein the IPIB/PIB is an osteoactive bisphosphonate.

20. The use according to any one of the preceding claims wherein the inhibitor is an analogue or derivative of an osteoactive PIB, for example a derivative having a group for promoting cellular uptake (e.g. a lipophilic group or a dipeptide group).

21. The use according to any one of the preceding claims wherein the inhibitor comprises a positively charged nitrogen atom.

22. The use according to any one of the preceding claims wherein the inhibitor is alendronate, pamidronate or ibandronate, or prenyl transferase inhibitory analogues or derivatives thereof.

23. The use according to any one of the preceding claims wherein the inhibitor is modified by the attachment of a lipophilic group or by the attachment of a dipeptide group permitting uptake via the cellular plasma membrane peptide carrier system.

24. Use of an inhibitor of IPI and/or prenyl transferase for the manufacture of a medicament for the regulation of bone metabolism, for example in the treatment of Paget's disease, hypercalcaemia (both tumour-induced and non-tumour induced), bone metastases or osteoporosis, wherein the inhibitor is not a geminal bisphosphonate.

25. A prenyl transferase and/or IPI inhibitor for use in therapy or prophylaxis, for example in the manufacture of a medicament for the regulation of bone metabolism (e.g. in the treatment of Paget's disease, hypercalcaemia (both tumour-induced and non-tumour induced), bone metastases or osteoporosis, wherein the inhibitor is not a geminal bisphosphonate.

26. The use of claim 24 or inhibitor of claim 25 wherein the inhibitor is an analogue of the carbocation:

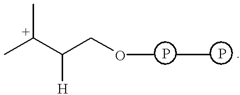

27. The use or inhibitor of any one of claims 24-26 wherein the inhibitor is a sulphonate, carboxylate, phosphonocarboxylate, phosphonosulphonate, phosphonophosphate or pyrophosphate.

28. The use or inhibitor of any one of claims 24-27 wherein the inhibitor comprises a positively charged nitrogen atom.

29. Use of IPI and/or prenyl transferase for binding a bisphosphonate, for example in an in vitro assay.

30. The use or inhibitor of any one of claims 24-29 wherein the IPI and/or prenyl transferase is one which, in vivo, mediates the physiological (e.g. antiresorptive) effects of osteoactive bisphosphonate.

31. The use or inhibitor of any one of claims 24-29 wherein the IPI and/or prenyl transferase is a Dictyostelium spp., for example Dictyostelium discoideum, IPI or prenyl transferase.

32. A method for screening for osteoactive drugs comprising the steps of:
(a) contacting a putative drug (e.g. a bisphosphonate) with the IPI and/or prenyl transferase as defined in any one of claims 24-31, and
(b) determining whether binding between the putative drug and IPI and/or prenyl transferase occurs, wherein binding is indicative of an osteoactive drug (e.g. an osteoactive bisphosphonate).

33. A method for evaluating the therapeutic activity of a putative drug (e.g. a bisphosphonate) comprising the steps of:
(a) contacting the drug with the IPI and/or prenyl transferase as defined in any one of claims 24-31, and either
(b) measuring the binding affinity of the putative drug for the IPI and/or prenyl transferase, or
(c) measuring the extent of inhibition imposed by the putative drug on IPI and/or prenyl transferase activity.

34. A method for synthesising a therapeutically active (e.g. antiresorptive or antiarthritic) drug (e.g. a bisphosphonate) comprising the steps of:
(a) providing a three-dimensional model comprising a catalytic site of the IPI and/or prenyl transferase as defined in any one of claims 24-31 (e.g. by computer analysis of its amino-acid sequence or by X-ray crystallography of the IPI and/or prenyl transferase or fragment thereof), and
(b) modelling the therapeutically active drug with reference to the three-dimensional model generated in step (a).

35. A therapeutically active drug (e.g. a bisphosphonate) which has been screened, evaluated or synthesised by the methods of claims 32 to 34.

36. An antibody (e.g. a monoclonal antibody) which binds (for example, specifically) to the IPI and/or prenyl transferase as defined in any one of claims 24 to 31.

37. A test kit comprising the IPI and/or prenyl transferase as defined in any one of claims 24-31, for example for use in the method of claims 32 or 33.

38. A test kit according to claim 37 wherein: (i) the IPI and/or prenyl transferase is bound to a solid support and/or (ii) the kit further comprises a labelled (e.g. radiolabelled or fluorescently labelled) bisphosphonate and/or (iii) the kit further comprises the antibody of claim 36.

38. A mimetic or antagonist of the IPI and/or prenyl transferase as defined in any one of claims 24-31, the mimetic for example consisting essentially of the bisphosphonate binding site of IPI and/or prenyl transferase.

39. The antibody of claim 36 or mimetic or antagonist of claim 38 for use in therapy, prophylaxis or diagnosis (e.g. for use as defined in claim 25 or in imaging).

40. A process for producing an osteoactive drug comprising the step of providing an analogue of the carbocation: 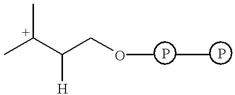
which analogue has a positively charged nitrogen atom in an alkyl chain thereof.

41. A process for selectively inhibiting an enzyme involved in sterol/isoprenoid biosynthesis comprising the step of providing an analogue of the carbocation: 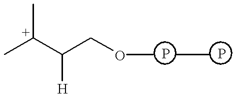
which analogue has a positively charged nitrogen atom in an alkyl chain thereof.

42. The process of claim 40 or 41 wherein the drug or inhibitor is not a bisphosphonate.

43. The process of claim 40 or claim 41 wherein the drug or inhibitor is a bisphosphonate (for example a geminal or non geminal bisphosphonate), a sulphonate, carboxylate, phosphonocarboxylate, phosphonosulphonate, phosphonophosphate or pyrophosphate.

44. The process of any one of claims 40-43 wherein the drug or inhibitor is further modified by the attachment of a lipophilic group or by the attachment of a dipeptide group permitting uptake via the cellular plasma membrane peptide carrier system.

45. A process for increasing the therapeutic efficacy of an osteoactive bisphosphonate comprising the step of introducing a positively charged nitrogen atom into an alkyl chain thereof.

46. A process for producing a therapeutically active (e.g. antiresorptive or antiarthritic) drug (e.g. a bisphosphonate) comprising the steps of:
(a) screening for a putative therapeutically active drug by: (i) contacting a putative drug (e.g. a bisphosphonate) with the IPI and/or prenyl transferase as defined in any one of claims 24-31, and (ii) determining whether binding between the putative drug and IPI and/or prenyl transferase occurs, wherein binding is indicative of an osteoactive drug (e.g. an osteoactive bisphosphonate);
(b) synthesising the screened drug of step (a) (or a derivative thereof).

47. A process for producing a therapeutically active (e.g. antiresorptive or antiarthritic) drug (e.g. a bisphosphonate) comprising the steps of:
(a) providing a three-dimensional model comprising a catalytic site of the IPI and/or prenyl transferase as defined in any one of claims 24-31 (e.g. by computer analysis of its amino-acid sequence or by X-ray crystallography of the IPI and/or prenyl transferase or fragment thereof), and
(b) modelling the therapeutically active drug with reference to the three-dimensional model generated in step (a); and
(c) synthesising the modelled drug of step (b) (or a derivative thereof).

48. The invention of any one of the preceding claims wherein the inhibitor is dimethylaminoethyl diphosphate.
